(19) United States
(12) Patent Application Publication
(10) Pub. No.: US 2010/0042439 A1
(43) Pub. Date: Feb. 18, 2010

(54) AUTONOMOUS PERPETUAL INVENTORY FOR HEALTHCARE

(75) Inventors: Pedro Martinez, Boca Raton, FL (US); Noel J. Guillama, Wellington, FL (US); Chester Heath, Boca Raton, FL (US)

Correspondence Address: COZEN O'CONNOR, P.C., 1900 MARKET STREET, PHILADELPHIA, PA, 19103-3508, US

(73) Assignee: THE QUANTUM GROUP, INC., Wellington, FL (US)

(21) Appl. No.: 12/543,060

(22) Filed: Aug. 18, 2009

Related U.S. Application Data
(60) Provisional application No. 61/089,581, filed on Aug. 18, 2008.

(51) Int. Cl. G06Q 50/00; G06Q 10/00

(52) U.S. Cl. 705/3

(57) ABSTRACT

An automated system for managing inventories of medical supplies at medical offices is disclosed wherein a database and intelligent computer system is used to anticipate the need for supplies based on appointments with patients, for example, so that inventories are maintained at optimum levels without the need for individuals to guess at which supplies to order at which particular times and a computer method for inventory management and replenishment comprising storing records of items of supplies and durable medical equipment at each medical office, clinic, and hospital subscribing to a management system, storing patient records comprising prior treatments, health diagnoses, and health status, storing tables of treatment and diagnostic codes corresponding to supplies and/or durable medical equipment required for each of said codes, wherein upon entry by a medical office, clinic, or hospital of a treatment or diagnostic event for a patient using a treatment or diagnostic code, looking up the corresponding patient record to determine whether a redundancy event has occurred and if no redundancy event is determined, issuing an order for the corresponding supplies and/or equipment.

### CROSS-REFERENCE TO RELATED APPLICATIONS

This application claims benefit of provisional application 61/089,581, filed Aug. 18, 2008, which is hereby incorporated by reference in its entirety.

### BACKGROUND OF THE INVENTION

This invention relates to the field of medical office management, more particularly to the field of management of medical inventories.

By “medical office,” we mean physician offices and practices, hospitals, and any other facility where patients are treated and inventories of medical supplies are maintained. Currently inventories of medical supplies are maintained in a medical office manually in the sense that when supplies are needed or about to run out, an individual in the medical office orders a resupply. No current system for medical offices allows for prediction of needs for medical supplies. By “medical supplies” we mean any type of consumable goods which is traditionally maintained in inventory in a medical office as well as durable medical equipment. Furthermore, no current system warns of unusual or excessive usage of medical supplies by a particular medical office or group of medical offices. No existing system provides automated ordering of medical supplies based on current and predicted consumption.

It has been suggested previously to maintain on a centralized database accessible over the Internet records of patient medical histories for large numbers of patients, perhaps all patients insured by a particular company or perhaps virtually all individuals in a country or in the world. One advantage of such a system is that the patient records are accessible from any location so that when the patient sees any medical provider, the record is immediately available to that medical provider. Another advantage of such a system is that the records can be analyzed to determine averages, trends, and norms, billing for patient-provider encounters, insurance coverage and collections can all be made more efficient by such centralized systems.

Such centralized systems can provide ubiquitous medical care for the individual insofar as records from all care providers can be consolidated in one database and made easily and instantly available over the Internet to any other care provider for the same person and anywhere on the planet.

To assure that all providers have fully interchangeable data, and that all data is uniformly collected, one way to accumulate the database is as part of an integrated offering that combines health records and practice management for medical offices.

It is an object of the present invention to improve on existing methods of managing inventory of medical supplies in medical offices. It is another object of the invention to automate ordering of medical supplies for use in a medical office based on current and predicted consumption. A still further object of the invention is to warn of unusual or excessive usage of medical supplies by a particular medical office or group of medical offices.

### SUMMARY OF THE INVENTION

These objects and others as will become apparent from the following disclosure are achieved by the present invention which comprises in one aspect an automated perpetual inventory of medical supplies which is maintained in a medical office as herein defined.

The invention comprises in another aspect a computer method for inventory management and replenishment comprising storing records of items of supplies and durable medical equipment in inventory at each of a plurality of medical offices subscribing to a management system, storing patient records comprising prior treatments, health diagnoses, and health status on a remote host computer system accessible from each of the plurality of medical offices over a computer network, storing tables of treatment and diagnostic codes corresponding to supplies and/or durable medical equipment required for each of said codes, wherein upon entry by a medical office, clinic, or hospital of a treatment or diagnostic event for a patient using a treatment or diagnostic code, looking up the corresponding patient record to determine whether a redundancy event has occurred and if no redundancy event is determined, issuing an order for the corresponding supplies and/or equipment.

The redundancy event in some embodiments is determined based on prior similar or identical treatment and/or diagnostic codes in the patient record, the relative times of said prior similar or identical treatments of diagnoses, and a standards module.

Supplies and/or medical equipment which are not in current inventory at the medical office can be automatically ordered immediately and supplies and/or medical equipment which are in current inventory at the medical office, clinic, or hospital can be ordered on a replenishment schedule which includes aggregating supplies and/or equipment requests, using the system of the invention.

Supplies and/or medical equipment utilized by previous doctors of a new patient whose record indicates an existing ailment can be automatically ordered prior to a patient visit.

Data from a plurality of medical offices, clinics, and/or hospitals can be processed to determine normal usage is for a supply, wherein treatments trends can be determined and reported, and wherein exceptional usage of supplies and/or medical equipment can be determined and reported.

Pilferage, waste, excessive prescription, or use of ineffective and/or redundant treatments for a patient or a medical office can be determined without a requirement for centralizing specific patient data and without a need for continuous communication.

Usage of supplies and/or equipment by a first medical office can be compared to usage by a by one or more other medical offices using data stored on a web server host which can be searched by CPT code, patient, and/or supply to determine excessive use, available alternatives, or applicable new or reduced cost products.

The invention comprises not only the above-described computer assisted method but a computer system comprising a web server, storage, and one or more CPUs, programmed to carry out the method, as well as an article comprising a set of computer readable instructions configured to carry out the method

In some embodiments a cache of transactions of lower priority such as inventory requirements, can be maintained locally in order to conserve Internet bandwidth so such bandwidth is available for more immediately important transactions, such as health records, during Internet outages or periods of congestion.

A copy of the medical supplies inventory and subset of patient needs can be maintained locally at the medical office as well as on an online host system assessable over the Internet network or in some embodiments may be maintained exclusively on the remote host system. The remote host system would be maintained by a service provider and would include algorithms for calculating usage by patient, malady, medical office, supplier, and possibly other categories such that medical supplies can be ordered in the most optimal way with regard to inventory can be maintained and to some degree usage can be predicted based on transactions with the service provider host computer.

The remote host system in some embodiments maintains a very large database, which is typically organized as an N-dimensional matrix—to which the inventory data, and patient data is added periodically. The inventory reporting, and consequential replenishment ordering, is normally parsed from this database on the host.

According to one feature of this invention, a local copy of a subset of the array is maintained on the doctors' local office system. Interactions, based on CPT code driven usage, would occur with this matrix and be periodically synchronized with the host system to avoid errors caused by host system, or connectivity outage. Data not available in the local matrix (e.g. a new patient) can be added to the local array from the host array as required.

According to a second feature of the invention, use of supplies is also organized by patient in the local and host arrays, such that unusual supplies can be pre-ordered to be available at the time of the patient's next scheduled visit. Specific supplies, utilized by previous doctors of a new patient, can be automatically ordered upon entry of an appointment event into the remote host and be on hand for new patients with existing ailments, an advantage to medical offices where the supplies probably needed for the new are not normally stocked by the new physician.

According to a third feature of the invention, based on usage of supplies by other medical offices, the arrays can be searched by CPT code, patient, supply or other attribute. For example, reports might include excessive use, available alternatives, or applicable new or reduced cost products.

This system and method of the invention provides efficiency, quality control and cost optimization to doctors' offices, hospitals and care providers in a uniform and productive means. It does not wait until the end of the month inventory analysis. The system and method provide instantaneous demand based on usage and projected demand based on trend information. The invention for the first time provides a real time, electronic system designed to coordinate medical supply needs based on immediate patient/doctor consumption and historical trends and usage patterns. The system and method creates a real time and dynamic means to optimize medical supply management. The efficiency of this system can generate improved productivity, cost reduction (optimized ordering, reduced shelf space, vendor management) and quality of supplies for doctors' offices, hospitals and care facilities. According to this invention, data from many medical offices is combined at a central host database allowing preordering of supplies according to an interactive intelligent system.

### BRIEF DESCRIPTION OF THE DRAWINGS

FIG. 1 is a flow chart of one aspect of the invention wherein a single ended process is shown.

FIG. 2 is a flow chart of an Automated Health Inventory Management System Process (AHIMS) according to the invention.

FIG. 3 is a flow chart of a single ended process according to the invention.

FIG. 4 is a flow chart of a System Interlock process according to the invention.

FIG. 5 is a flow chart of a perpetual inventory process according to the invention.

FIG. 6 is a block diagram illustrating components of a system according to the invention.

### DETAILED DESCRIPTION OF THE INVENTION

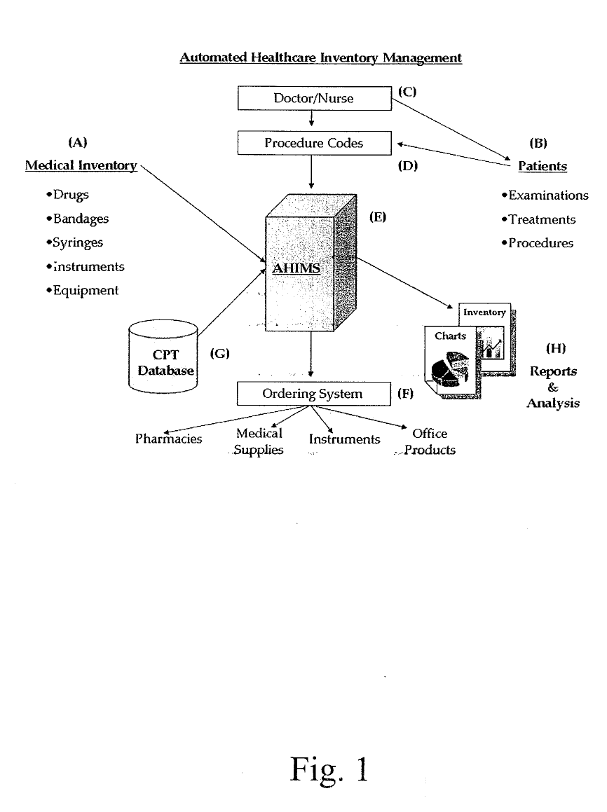

Referring first to FIG. 1, a flow chart illustrating one embodiment of the invention is shown wherein a database of records of existing inventory (A) is resident in a local computer in a medical office. Patients (B) with a variety of conditions receive (C) a series of treatments by doctors and nurses at the medical office. In each case the doctor enters a Current Procedural Terminology (CPT) code for the treatment into the local computer and the data is sent over the Internet on a real time basis or periodically to the remote host maintained by the service provider. CPT codes are available from agency databases (G). The supplies required for the procedures and treatments (i.e., drugs, bandages, syringes, specialized equipment, etc.) (A) are consumed at the time of treatment or are provided for the patients to take with them.

The doctor (D) documents the procedures and treatments performed on the patient using a CPT code or its equivalent. Each procedure has a typical number of supporting supplies associated with the treatment. The CPT code (or equivalent), patient record and inventory stocking are matched in the AHIMS (E). A re-stocking order is generated and aggregated into the ordering system (F) at the doctor's office, hospital or care facility. In parallel, the AHIMS generates automatic reports on usage, trends and analysis (H).

The doctor's office ordering system generates the orders for supplies from pharmacies, medical supply companies, instrument distributors and office supply channels. The inventory is replenished and logged into the management system. An alternative means to the logging of new inventory is through bar code scanning or radio frequency identification (RFID) technology. This method provides instant recognition of new or actual inventory electronically.

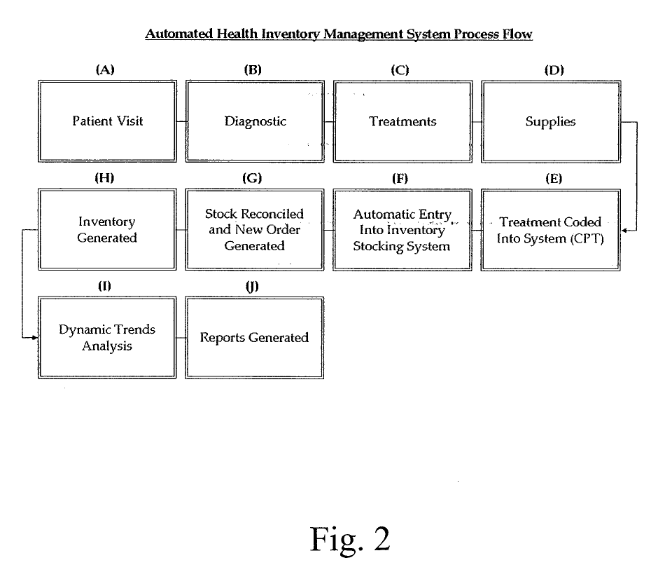

Referring now to FIG. 2, a process is illustrated which begins with a patient visit (A). A diagnostic procedure (B) is carried out by a medical professional. Treatments may be instituted (C), in which case supplies (D) may be used. The treatment is coded (E) by CPT codes into the system, which according to a novel feature of the invention is automatically entered into the inventory stocking system (F). Existing stock is compared to optimum stock levels (G) by a reconciliation process and if additional inventory is needed an inventory order is generated (H) or a report is generated (J) advising of need for medical supplies or equipment. The host system uses the treatments coded into the system to calculate trends dynamically (I) and reports on such trends can be generated. Among the report may be flagging of possible pilferage, misuse of medical supplies, waste, and lower cost alternatives.

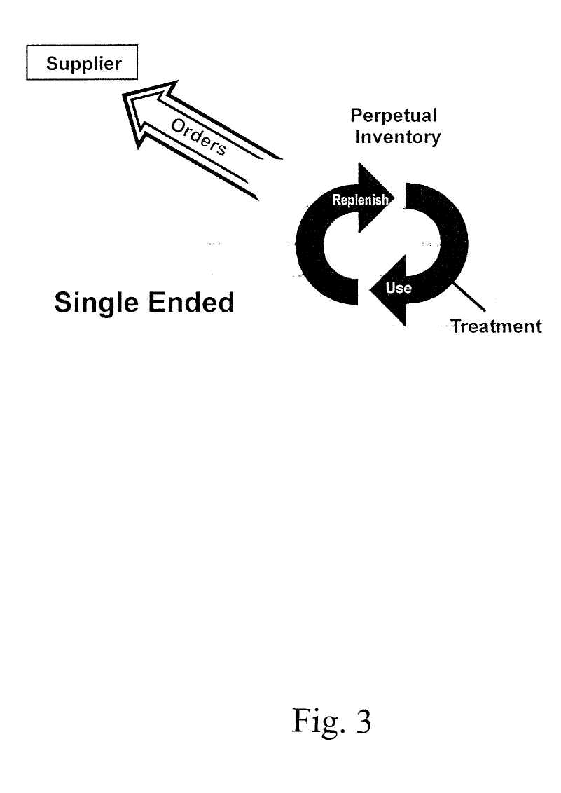

Referring to FIG. 3, the system receives treatment information and calculates use of medical supplies based on the treatment codes, and then determines the optimum amount, timing, and level of replenishment of the medical supplies. Upon determination of need for replenishment of medical supplies, the system generates orders which are sent automatically to the most appropriate suppliers.

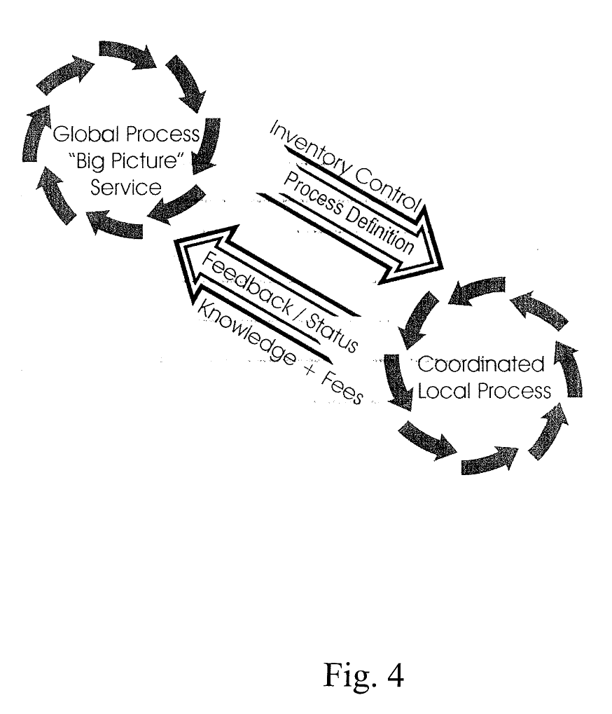

As illustrated in FIG. 4, there is a local process at local computers at each medical office and a host computer system called “global process big picture service” which communicates over the Internet with the local processes to send inventory control process definitions and receive feedback on status, knowledge, and fees. The process includes a Secure System interlock—not dissimilar to standard download processes, for example, iTunes, to assure connection is maintained and secure. This system operates normally will all traffic to the global process, except where Internet congestion may force lesser priority activities, such as perpetual inventory, to be done locally to preserve bandwidth for higher priority medical record transactions.

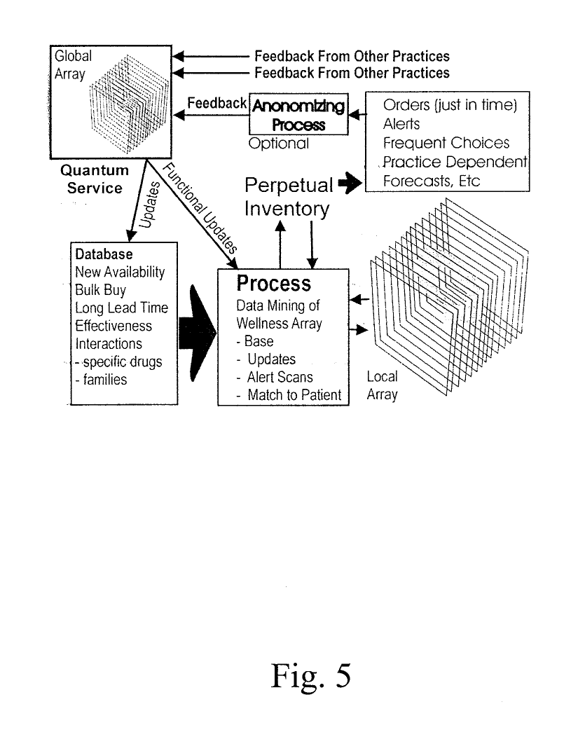

Referring now to FIG. 5 a remote host computer, also referred to as a Global array, which includes storage, processor, and database is provided. Also provided are local arrays at each of many medical offices.

The host computer functions as an automated central procurement service and is programmed to store inventory records of each of a plurality of medical offices, tables of CPT codes and associated medical supply requirements, and patient health records including prior treatment, medical conditions, insurance information, and virtually everything else related to the patients' health. For example first out (consumed)/first in (replenished) or another example per urgency—say here one bandage can be substituted for another with no ill effect, but an antibiotic might have no alternative so it should be replenished first. Another algorithm might be to limit total expenditure only for expensive items by maintaining less on hand, or requiring approval, while inexpensive items are replenished without approval.

The Global Array sees all orders, detected alerts, frequent choices and practice dependent information. Global Array information can be a simple aggregation of all local arrays or an anonymous subset.

The Perpetual Inventory System automatically orders supplies by CPT code, or as used. A process examines the Local Array for correlations to the order process. The process may create alerts if excessive use of one supply occurs relative to patient use records and treatment applied (e.g. disproportionate prescription of a drug listed in the database, excessive supplies used for a given procedure indicating waste or inventory shrinkage, frequent choices after alerts on poor effectiveness of treatment or drug).

According to a process controlled by the Service provider service, all orders are examined and management alerts created. Local adjustment of the process or database can be limited so as to assure independent automated oversight.

The local enterprise is not restricted from operation if communication with Service provider is lost, but the process and database can be adjusted when communication is restored.

Alerts can be statistical in nature (e.g. “you are using too many rubber gloves for a psychiatric practice”).

Alerts can also be patient specific and immediate, based on the local array, should interactions or redundant treatment be detected—without need for continuous communication with the Service provider Service.

The process learns from multiple practices what normal usage is for a given drug or supply and can alter the local process and database when an improper trend is detected.

Objective is to detect pilferage, waste, excessive prescription, or use of ineffective and/or redundant treatments without a requirement for centralizing specific patient data—and without a need for continuous communication.

Medical professionals still have full freedom of action, but with automated oversight.

Of course the concept can be generalized where global data is not anonymous. All data could be centralized and all examination of data is concentrated to one array. The service may be reduced to alerts to the enterprise only and updates to the database and process may be altered locally as well.

The protocol wherein the central procurement service pushes an algorithm to local data bases in medical offices and periodically updates a central database by synchronizing the cumulative requirement from a plurality of local databases, thereby permitting different reordering algorithms for different local offices—based on a central procurement plan. The organization of the coordinated arrays is unique. The proprietary protocols and organization could control portability of the enterprise local array to other services. From a business perspective, the Service provider Service can be shown to be cost justified based on its independent ability to control expenses and errors.

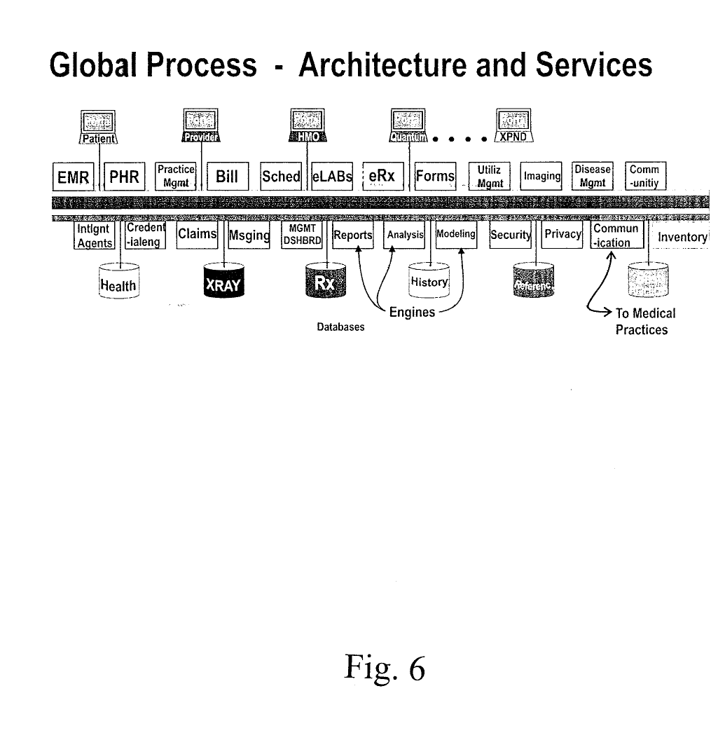

Referring to FIG. 6, a block diagram of an embodiment of a system of the invention is illustrate wherein the global process would normally be handling a multitude of practice management transactions involving appointment scheduling, patient billing, claims to payers, including inventory management of the practice office. Other more critical and immediate operations would be electronic medical records, transmission of prescription and laboratory orders, medical imaging storage, and disease management. Other background operations, such as security, communication, analysis modeling and the generation of reports occur in the global process to support of the other operations.

The typical interface to the medical provider's practice office would be as a dumb terminal, with the global process provided centrally by a server on the Internet which maintains databases for all of the operations, typically in the format of arrays as per the description of FIG. 5. In periods of unavailability or congestion, these databases may not be instantly available over the web and some portion is maintained as an array subset locally to be transmitted and synchronized with the Global Array referred to in FIG. 5 as the “Quantum Service” which is a trade name for a service provider.

Normally, keystrokes on the terminal in the medical office would be transmitted in a “dumb” fashion, character by character, with no processing to the Global service and would enter the process through the communication port. There, the data would be federated or made of equivalent units with similar data in the rest of the system and processed per the operation in progress—for example and inventory usage. The inventory usage would enter the global array and a simultaneous process would gather all inventory usage into a file for the medical provider's practice office and for a general combined order for group purchasing. Records of the status would be maintained in the global array as well.

In periods of limited Internet availability, a subset array, similar in concept and structure to the Global array, but smaller in size is maintained in the medical provider's practice office for later delivery to the Global array. In this case, the local process is aware that no acknowledgement of transactions is occurring with the global process and that limited Internet availability exists. It is considered obvious that other transactions of lesser productivity, such as billing or scheduling could be managed similarly in order to conserve bandwidth for the higher priority (e.g. medical health records) transactions.

The present invention, therefore, is well adapted to carry out the objects and attain the ends and advantages mentioned, as well as others inherent therein. While the invention has been depicted and described and is defined by reference to particular preferred embodiments of the invention, such references do not imply a limitation on the invention, and no such limitation is to be inferred. The invention is capable of considerable modification, alteration and equivalents in form and function, as will occur to those ordinarily skilled in the pertinent arts. The depicted and described preferred embodiments of the invention are exemplary only and are not exhaustive of the scope of the invention. Consequently, the invention is intended to be limited only by the spirit and scope of the appended claims, giving full cognizance to equivalents in all respects.

What is claimed is:

1. A computer method for inventory management and replenishment comprising storing records of items of supplies and durable medical equipment in inventory at each of a plurality of medical offices subscribing to a management system, storing patient records comprising prior treatments, health diagnoses, and health status on a remote host computer system accessible from each of the plurality of medical offices over a computer network, storing tables of treatment and diagnostic codes corresponding to supplies and/or durable medical equipment required for each of said codes, wherein upon entry by a medical office, clinic, or hospital of a treatment or diagnostic event for a patient using a treatment or diagnostic code, looking up the corresponding patient record to determine whether a redundancy event has occurred and if no redundancy event is determined, issuing an order for the corresponding supplies and/or equipment.

2. The computer method of claim 1 wherein the redundancy event is determined based on prior similar or identical treatment and/or diagnostic codes in the patient record, the relative times of said prior similar or identical treatments of diagnoses, and a standards module.

3. The computer method of claim 1 wherein supplies and/or medical equipment which are not in current inventory at the medical office, clinic, or hospital are ordered immediately and supplies and/or medical equipment which are in current inventory at the medical office, clinic, or hospital are ordered on a replenishment schedule which includes aggregating supplies and/or equipment requests.

4. The computer method of claim 1 wherein supplies and/or medical equipment utilized by previous doctors of a new patient whose record indicates an existing ailment are ordered prior to a patient visit.

5. The computer method of claim 1 wherein data from a plurality of medical offices, clinics, and/or hospitals is processed to determine normal usage is for a supply, wherein treatments trends can be determined and reported, and wherein exceptional usage of supplies and/or medical equipment can be determined and reported.

6. The computer method of claim 1 wherein pilferage, waste, excessive prescription, or use of ineffective and/or redundant treatments for a patient or a medical office are determined without a requirement for centralizing specific patient data and without a need for continuous communication.

7. The computer method of claim 1 wherein usage of supplies and/or equipment by a first medical office is compared to usage by a by one or more other medical offices using data stored on a web server host which can be searched by CPT code, patient, and/or supply to determine excessive use, available alternatives, or applicable new or reduced cost products.

8. The computer method of claim 1 wherein the network is the Internet.

9. A computer system comprising a web server, storage, and one or more CPUs, programmed to carry out the method of claim 1.

10. An article comprising a set of computer readable instructions configured to carry out the method of claim 1.

11. A remote cache of lesser important transactions, such as inventory requirements to be maintained locally in order to conserve Internet bandwidth for more immediately important transactions, such as health records, during Internet outages or periods of congestion, configured to carry out the method of claim 1.
